(19) United States
(12) Patent Application Publication
(10) Pub. No.: US 2008/0109023 A1
(43) Pub. Date: May 8, 2008

(54) Method and apparatus for altering the appearance of a patient's skin

(76) Inventor: Steven E. Greer, New York, NY (US)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 11/801,393

(22) Filed: May 9, 2007

Related U.S. Application Data
(60) Provisional application No. 60/856,661, filed on Nov. 3, 2006.

(51) Int. Cl. A61B 17/32

(52) U.S. Cl. 606/170

(57) ABSTRACT

A dissecting device includes a plurality of blades which are pivotally mounted on a base. A wire is connected with each of the blades to effect pivotal movement of the blades relative to the base. The base includes a guide wire passage. When the appearance of a patient's skin is to be changed, the dissecting device is moved onto a guide wire which has been inserted beneath a selected portion of the patient's skin. The dissecting device is moved along the wire to a location beneath the selected portion of the skin and is actuated to dissect connective tissue. An implant, such as a collagen sheet, mesh, or other material, is positioned at the location beneath the selected portion of the patient's skin.

### RELATED APPLICATIONS

This application claims priority to U.S. Provisional Patent Application Ser. No. 60/856,661 Filed Nov. 3, 2006 by Steven E. Greer. The benefit of the earlier filing date of the aforementioned application is hereby claimed. The disclosure in the aforementioned U.S. Provisional Patent Application Ser. No. 60/856,661 is hereby incorporated herein its entirety by this reference thereto.

### BACKGROUND OF THE INVENTION

Cosmetic surgery has been performed to alter the appearance of a portion of a patient's skin. Thus, cosmetic surgery has been performed to minimize nasolabial lines, squint lines or crows' feet, forehead or worry lines, neck lines, and/or cheek lines. Methods and devices for performing cosmetic surgery to eliminate or minimize facial lines are disclosed in Published U.S. Patent Application Nos. 2006/0161253 and 2005/0055073.

### BRIEF SUMMARY OF THE INVENTION

The present invention relates to a method of altering the appearance of a selected portion of a patient's skin. The method includes inserting a portion of a wire beneath the selected portion of the patient's skin and moving a dissecting device along the wire to a location beneath the selected portion of the patient's skin. Connective tissue which connects the selected portion of the patient's skin with the tissue disposed beneath the patient's skin, is cut with the dissecting device. After the connective tissue has been cut, an implant is positioned at the location beneath the selected portion of the patient's skin.

The dissecting apparatus may include a plurality of blades which are pivotally connected to a base. The blades are connected with actuator wires which are movable to effect pivotal movement of the blades relative to the base. The base may include a passage which receives a guide wire to guide movement of the base relative to the patient's body tissue.

### BRIEF DESCRIPTION OF THE DRAWINGS

The foregoing and other features of the invention will become more apparent upon a consideration of the following description taken in connection with the accompanying drawings wherein:

FIG. 1 is a top plan view of a dissecting device;

FIG. 2 is a side elevational view, taken generally along the line 2-2 of FIG. 1, further illustrating the construction of the dissecting device;

FIG. 3 is a front elevational view, taken generally along the line 3-3 of FIG. 2, further illustrating the construction of the dissecting device;

FIG. 4 is a fragmentary schematic illustration depicting the manner in which a hollow needle is inserted beneath a selected portion of a patient's skin;

FIG. 5 is a fragmentary schematic illustration, generally similar to FIG. 4, illustrating the manner in which the guide wire is inserted through the hollow needle;

FIG. 6 is a fragmentary schematic illustration depicting the manner in which the dissecting device of FIGS. 1-3 is moved along the guide wire of FIG. 5 to a location beneath a selected portion of a patient's skin;

FIG. 7 is a schematic illustration depicting the manner in which the dissecting device of FIGS. 1-3 is operated from a retracted condition to an extended condition when the dissecting device is disposed beneath a selected portion of the patient's skin;

FIG. 8 is a fragmentary schematic illustration depicting the manner in which the dissecting device is moved along the guide wire, after the dissecting device has been moved to the extended condition of FIG. 7, to dissect connective tissue;

FIG. 9 is a schematic illustration depicting the manner in which a nasolabial crease or line is reduced by cutting connective tissue disposed beneath the crease;

FIG. 10 is a fragmentary schematic illustration depicting the manner in which an implant introducer may be moved along the guide wire to position an implant beneath the selected portion of a patient's skin with the implant in a retracted condition;

FIG. 11 is a schematic illustration depicting the implant in a retracted condition inside the introducer;

FIG. 12 is a schematic illustration depicting the manner in which the implant is expanded by the introducer;

FIG. 13 is an enlarged schematic illustration depicting an implant in a partially expanded condition; and

FIG. 14 is a schematic illustration, generally similar to FIG. 13, depicting the implant in a fully expanded condition.

### DESCRIPTION OF SPECIFIC EMBODIMENTS OF THE INVENTION

### Dissecting Device

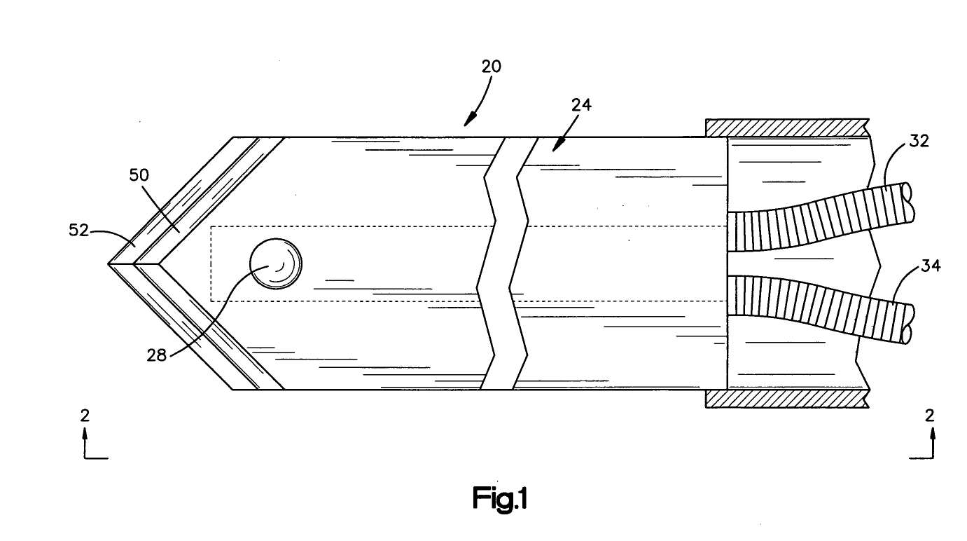

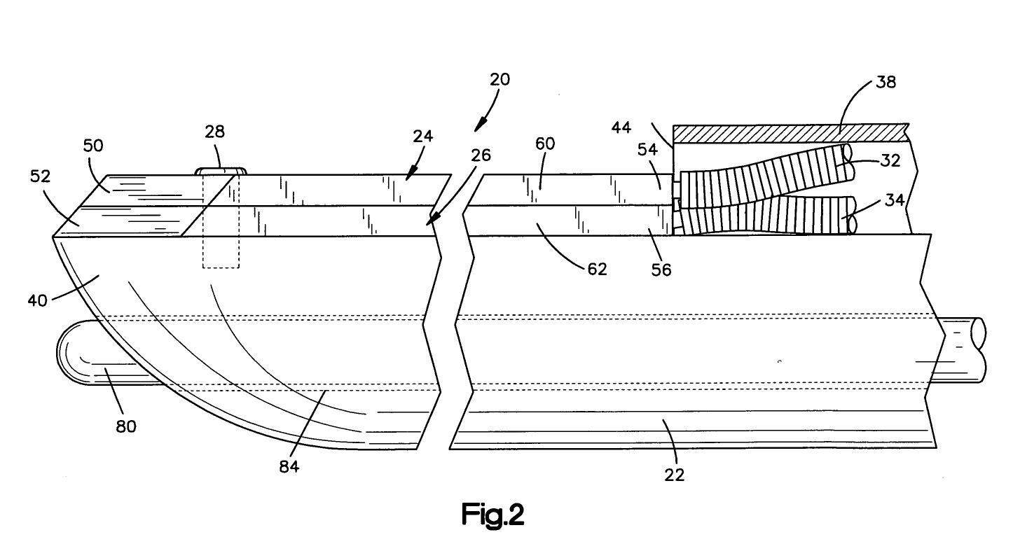

A dissecting device 20 is illustrated in FIGS. 1-3. The dissecting device 20 includes a metal base 22 on which upper and lower metal blades 24 and 26 are pivotally mounted by a pivot pin 28. Push wires 32 and 34 are connected to the trailing or left (as viewed in FIG. 2) ends of the blades 26.

The push wires 32 and 34 transmit force to pivot the blades 24 and 26 relative to the base 22. This pivotal movement of the blades 24 and 26 moves them from the retracted condition illustrated in FIGS. 1-3 to an extended condition illustrated in FIG. 7. A tubular housing or dome 38 is fixedly connected to the base. The dome 38 extends over the push wires 32 and 34 so that they are not exposed to a patient's body tissue, except at the locations where they are connected with the blades 24 and 26. If desired, the entire dissector device may be enclosed by the tubular housing or dome 38.

The leading end 44 of the tube 38 may enclose the portion of the base 22 and the blades 24 and 26 rearwardly of the central axis of the pivot pin 28. Once the dissecting device 20 has been positioned in a patient's body, the tube or housing 38 would be withdrawn so that the tubular housing 38 would be disposed rearwardly of the trailing ends of the blades 24 and 26, in the manner illustrated in FIG. 2. This would result in the blades being disposed so that they would be free to pivot to the extended condition of FIG. 7.

The pivot pin 28 extends through leading end portions 50 and 52 of the blades 24 and 26. When the blades 24 and 26 are to be moved from the retracted condition of FIGS. 1-3 to the extended condition of FIG. 7, the push wires 32 and 34 pivot the blades 24 and 26 about the pivot pin 28.

Since a large majority of the length of the blades 24 and 26 is disposed rearwardly of the pivot pin 28, trailing end portions 54 and 56 (FIG. 2) of the blades 24 and 26 pivot outward, in opposite directions, from the base 22. As the trailing end portions of the blades 24 and 26 move away from each other, blunt side surfaces 60 and 62 on the blades 24 and 26 dissect the body tissue. The blunt side surface 60 and 62 press against the body tissue and separate the body tissue to form a planar space in the body tissue. It should be noted that space is formed in the body tissue by the portions of the blade 24 and 26 which are disposed rearward, that is toward the right, as viewed in FIG. 2, of the pivot pin 28.

Although it is believed that it may be desired to have the blades 24 and 26 with relatively blunt side surfaces 60 and 62, the blades 24 and 26 may be formed with sharp edges to cut the body tissue as well as to push against the body tissue. Thus, the blades may be configured so as to provide just a pushing action against body tissue as the blades are pivoted about the pin 28. Alternatively, the blades may be provided with sharp edges which provides a cutting action as well as a pushing action as the blades are pivoted about the pin 28. The pushing and/or cutting action occurs at a location which is rearward, toward the right as viewed in FIG. 2, of the pivot pin 28.

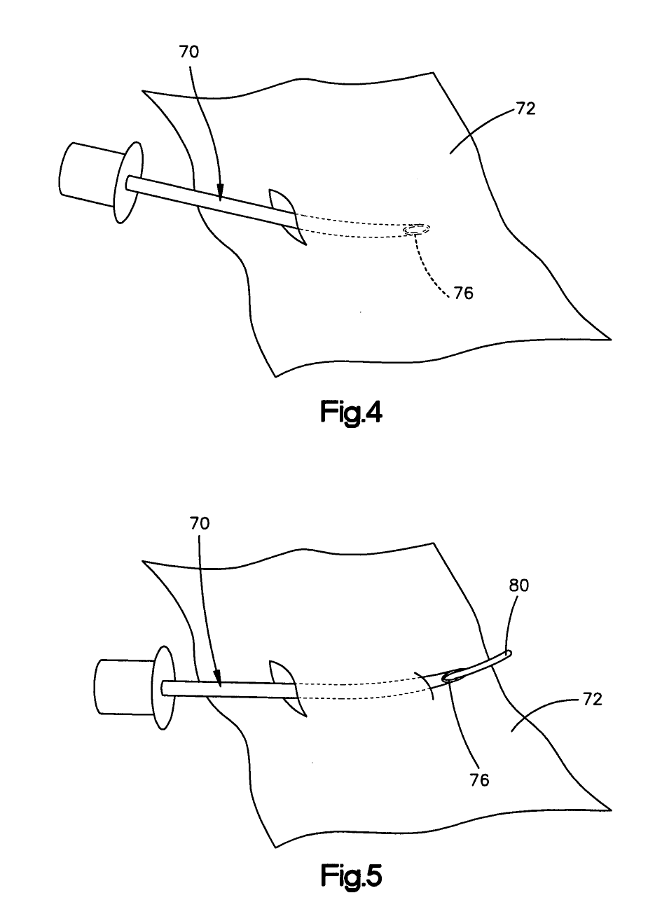

When the dissecting device 20 is to be used to minimize a crease or line in a patient's face, such as a nasolabial crease, a hollow needle 70 (FIG. 4) is inserted through a patient's skin 72 adjacent to the crease. The hollow needle 70 is flexible so that it can be guided to a desired orientation relative to the patient's skin 72. Alternatively, the hollow needle 70 may have a relatively rigid configuration which is maintained while it is being inserted into the patient's skin 72. For example, the hollow needle 70 may have an arcuately curving configuration so that a leading end portion 76 of the hollow needle 70 moves along an arcuate path relative to the patient's body. The hollow needle 70 is then moved to a desired position relative to the patient's skin. The leading end portion 76 of the hollow needle emerges from the patient's skin in the manner illustrated schematically in FIG. 5.

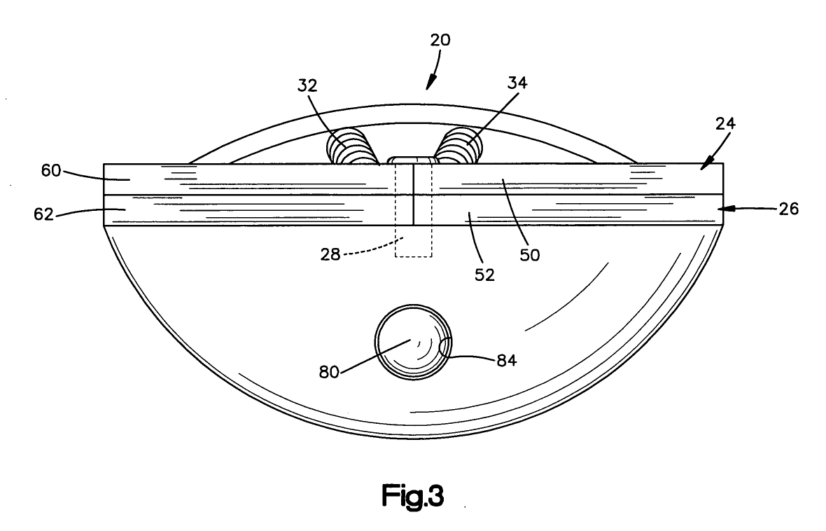

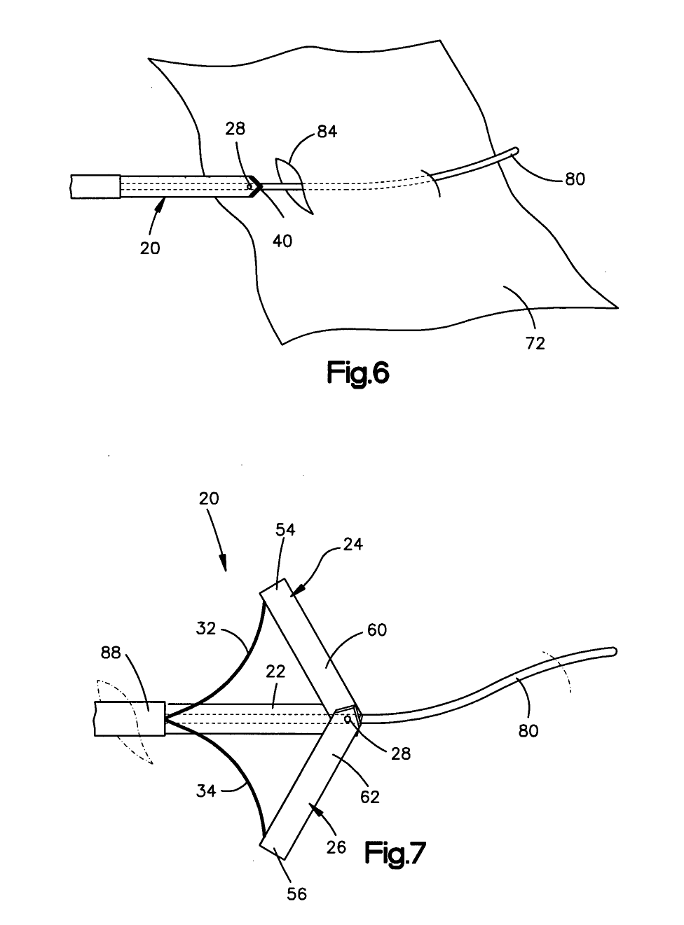

A flexible guide wire 80 is then inserted through the hollow needle 70. Once the flexible guide wire 80 has been inserted through the hollow needle, the needle is withdrawn from the flexible wire. The flexible wire 80 is then inserted through a passage 84 (FIGS. 2 and 3) in the base 22 of the dissecting device 20. The retracted dissecting device 20 slides along the guide wire 80 and enters the patient's body tissue through an opening 84 in the patient's skin 72 (FIG. 6). The leading end portion 40 of the dissecting device 20 is tapered so that it pushes aside body tissue as the dissecting device moves along the guide wire to a location beneath the patient's skin 72.

When the dissecting device 20 has moved to a desired location relative to the patient's skin 72, the dissecting device is operated from the retracted condition of FIGS. 1-3 to the extended condition of FIG. 7. To operate the dissecting device 20 to the extended condition of FIG. 7, the push wires 32 and 34 transmit force to trailing end portions 54 and 56 of the upper and lower blades 24 and 26. This force pivots the blades 24 and 26 in opposite directions about the pivot pin 28. As this occurs, the side surfaces 60 and 62 on the blades 24 and 26 push body tissue aside to initiate formation of a planar discontinuity or separation in the body tissue at a location beneath the skin 72.

The pushing action of the blades 24 and 26 against the body tissue is effective to sever or rupture connective tissue, such as ligaments at the location where formation of the planar discontinuity is initiated by operation of the dissecting device 20 from the retracted condition to the extended condition. The dissecting device 20 is then moved along the guide wire 80 to increase the extent of the planar separation which is formed in the patient's body tissue at a location beneath the facial crease which is to be minimized. The extent of the planar separation which is formed in the patient's body tissue will depend upon the extent of the crease or line which is to be minimized.

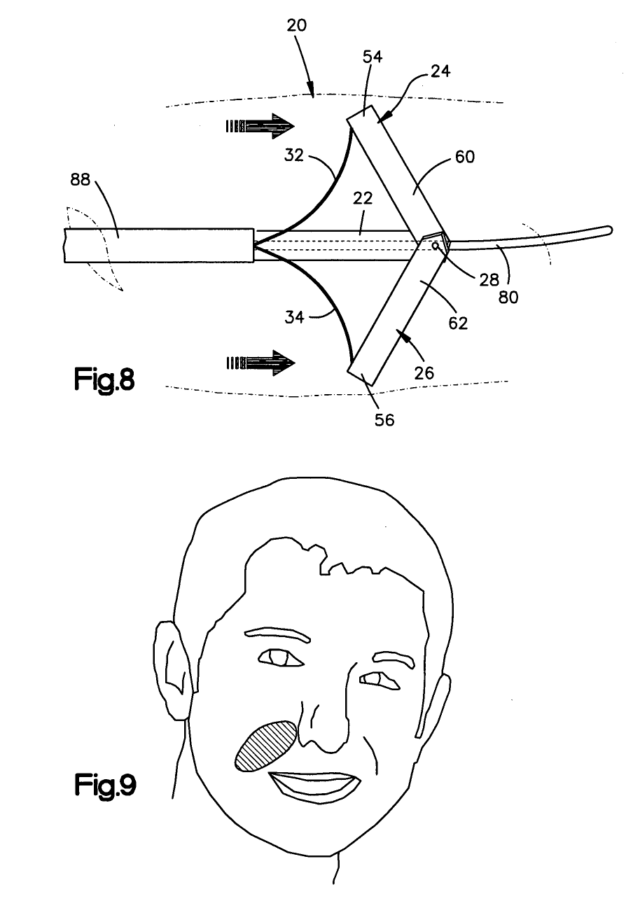

The manner in which the extended dissecting device 20 is moved along the guide wire 80 has been illustrated schematically in FIG. 8. The base 22 of the dissecting device 20 is connected with a flexible tube 88 which pushes the base, and the extended blades 24 and 26 along the guide wire 80 to form a planar separation having a central axis which corresponds with the central axis of the guide wire 80.

Although it is believed that it may be desired to have the relatively blunt leading side surfaces 60 and 62 of the blades 24 and 26 push aside body tissue to form the planar discontinuity or separation, the blades may be configured so as to have relatively sharp edges which form the planar separation with a cutting action. If desired, a combination of a cutting action and pushing or rupturing action may be utilized to sever ligaments or other connective tissue interconnecting the patient's skin and the underlining body tissue, that is, the SMAS (superficial musculoaponeurotic system). Thus, the blades 24 and 26 are effective to sever multiple fibrous extensions which attach the underlying body tissue to the superficial epidermal-dermal portion of the skin in which the crease, such as the nasolabial line, is formed. Rupturing and/or cutting of the fibrous connective tissue between the skin and the underlying muscle layer beneath the skin forms a plane. Where this plane is formed, the outer layer of skin is disconnected from the underlying body tissue. This results in a minimization of lines, such as the nasolabial line, in the manner illustrated schematically in FIG. 9.

Once a plane or space having a desired extent is formed between the SMAS and the skin 72 of the patient, the push wires 32 and 34 are retracted to pivot the blades 24 and 26 from the extended condition of FIGS. 7 and 8 back to the retracted condition of FIGS. 2-3 and 6. The dissecting device 20 is then moved back along the guide wire 80 and withdrawn through the opening 84 in the patient's skin 72. Although the dissecting device 20 is withdrawn from the patient's body tissue, the guide wire 80 is left in position extending beneath the patient's skin and along the plane formed by the dissecting device 20.

After the planar separation has been established beneath the patient's skin 72, an implant is positioned in the planar separation to maintain the desired configuration of the patient's skin. The implant may be of a natural or artificial material. For example, the implant may be formed of collagen. Alternatively, the implant may be formed of any desired biocompatible material. The implant may be formed of a knit fibers, woven fabric, compressible or non compressible sponge material, closed or open cell foam, or other known implant materials.

Regardless of what material is utilized to form the implant, an implant introducer 92 (FIGS. 10-14) may be utilized to position the implant in the plane which was previously formed by the dissecting device 20. The implant introducer moves along the guide wire 80 in the same manner as in which the dissecting device 20 moved along the guide wire.

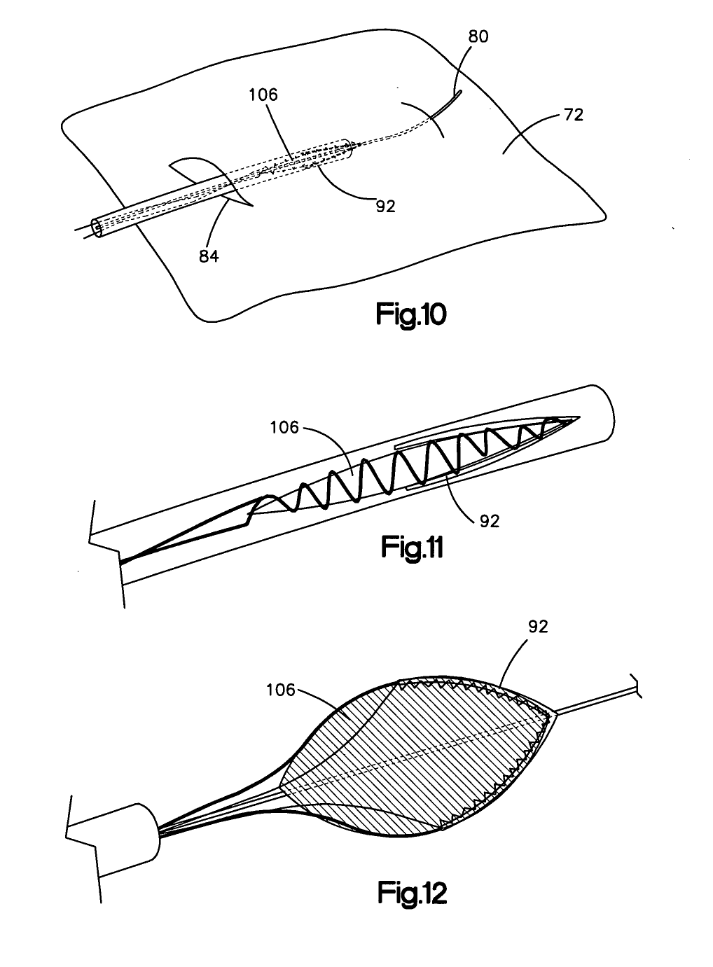

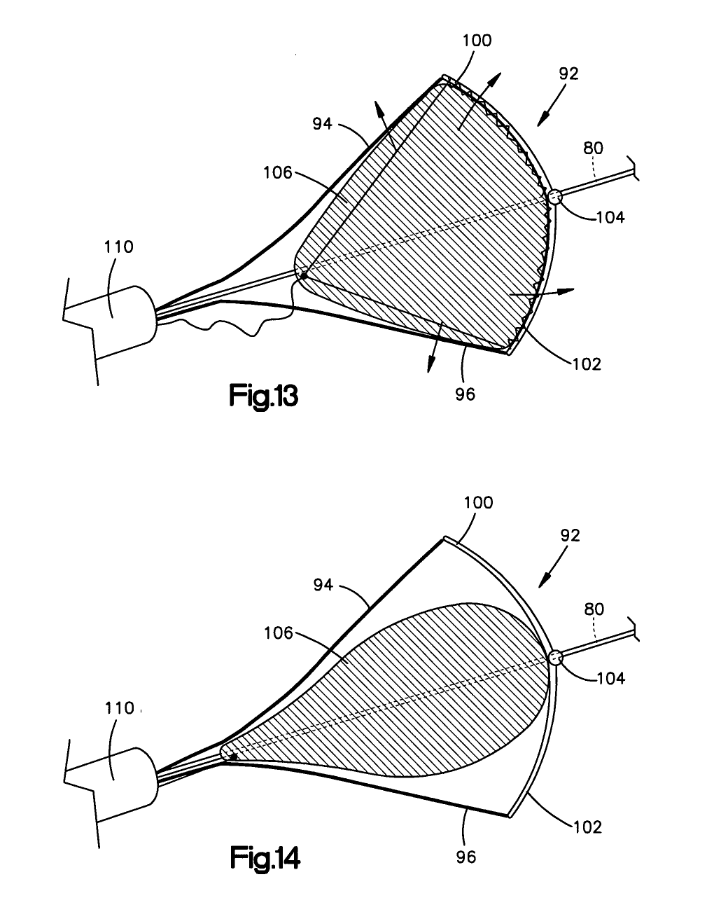

When the implant introducer is initially moved through the opening 84 (FIG. 10) in the patient's skin 72, the implant introducer 92 is in the retracted condition illustrated schematically in FIGS. 10 and 11. When the implant introducer 92 has been moved to a desired position relative to the patient's skin 92, the implant introducer is expanded in the manner illustrated in FIGS. 12 and 13. Push wires 94 and 96 (FIG. 13) are utilized to move arms 100 and 102 about a pivot connection 104. The arms 100 and 102 may be formed of metal and bent from the retracted condition of FIGS. 10 and 11 to the extended condition of FIG. 14. If desired, the arms 100 and 102 may be formed of Nitinol™. Alternatively, a pivot pin, similar to the pivot pin 28 (FIGS. 1-3), may be utilized to interconnect the arms 100 and 102.

As the arms 100 and 102 are moved to their extended condition (FIG. 14), the implant 106 is moved from a compact initial condition (FIGS. 10 and 11) to the extended condition of FIG. 14. As this occurs, the arms 100 and 102 pull the implant 106 and spread it outward along the plane which was previously established in the patient's body tissue by the dissecting device 20. Once the implant 106 has been moved to the extended condition of FIG. 14, the implant is released from the arms 100 and 102.

It is contemplated that the implant 106 may be released from the arms in any one of many different ways. For example, one or more grippers may be utilized to secure the implant 106 to the arms 100 and 102. These grippers will then be actuated to a release condition so that the arms can be moved relative to the extended implant 106. Alternatively, the implant 106 may be secured to the arms 100 and 102 by a filament. This filament may be withdrawn, that is moved toward the left as viewed in FIG. 14, and pulled free of the arms 100 and 102 to release the implant from the arms. Of course, a plurality of filaments may be utilized to interconnect the implant and the arms.

Once the implant 106 has been release from the arms 100 and 102, the implant introducer 92 is operated from the extended condition of FIG. 14 back to the retracted condition of FIGS. 10 and 11. This may be done by moving an introducer sheath 110 (FIG. 14) axially along the push wires 94 and 96. As this is done, the introducer sheath would be moved beneath the trailing or leftward (as viewed in FIG. 14) end portion of the implant 106. The arms 100 and 102 would be moved from the extended position of FIG. 14 back to the retracted positions (FIGS. 10 and 11) as they move into the introducer's sheath along the guide wire 80 to a location outside of the patients body.

Although only the elimination of a facial crease (nusolabial line) has been illustrated in FIG. 9, it is contemplated that the present invention may be utilized in association with skin on any desired part of a patient's body. For example, the method and apparatus of the present invention may be utilized to reduce or eliminate depressions which occur on the back of a patients hand at a location between extensor tendons on the dorsum of the patient's hand and wrist. When this is to be done, implant 106 may have a long narrow configuration corresponding to the configuration of the depression on the back or dorsum of the hand.

The method and apparatus of the present invention may be utilized to eliminate dimpling of the skin caused by protrusions of subcutaneous fat (cellulite). The objectionable dimpling cellulite may occur in skin on a patient's torso, legs, and/or arms. When the dimpling cellulite is to be eliminated, the dissecting device 20 is utilized to cut, push, and/or rupture subcutaneous connective tissue connected with the patient's skin. An implant 106 having a configuration corresponding to the configuration of an area of the patient's skin where the dimpling is to be eliminated, is then inserted beneath the patient's skin in the planar separation formed by the dissecting device 20.

### CONCLUSION

In view of the foregoing description, it is apparent that the present invention relates to a method of altering the appearance of a selected portion of a patient's skin 72. The method includes inserting a portion of a wire 80 beneath the selected portion of the patient's skin 72 and moving a dissecting device 20 along the wire to a location beneath the selected portion of the patient's skin. Connective tissue which connects the selected portion of the patient's skin 72 with the tissue disposed beneath the patient's skin, is cut with the dissecting device 20. After the connective tissue has been cut, an implant 106 is positioned at the location beneath the selected portion of the patient's skin.

The dissecting apparatus 20 may include a plurality of blades 24 and 26 which are pivotally connected to a base 22. The blades 24 and 26 are connected with actuator wires 32 and 34 which are movable to effect pivotal movement of the blades relative to the base 22. The base 22 may include a passage which receives a guide wire 80 to guide movement of the base relative to the patient's body tissue.

Having described the invention, the following is claimed:

1. A method of altering the appearance of a selected portion of patient's skin, said method comprising the steps of inserting a portion of a wire beneath the selected portion of the patient's skin, moving a dissecting device onto the wire, moving the dissecting device along the wire to a location beneath the selected portion of the patient's skin, and separating connective tissue which connects the selected portion of the patient's skin with tissue disposed beneath the patient's skin, said step of separating connective tissue includes moving blades in the dissecting device relative to the connective tissue.

2. A method as set forth in claim 1 further including the step of moving an implant along the wire to the location beneath the selected portion of the patient's skin and expanding the implant while at least a portion of the implant is at the location beneath the selected portion of the patient's skin.

3. A method as set forth in claim 1 wherein said step of inserting a portion of a wire beneath the selected portion of the patient's skin includes inserting the portion of the wire beneath a crease in skin on the patient's face, said step of separating connective tissue includes separating connective tissue disposed beneath the crease in the skin on the patient's face, said method further includes moving an implant along the wire to a location disposed beneath the crease in skin on the patient's face.

4. A method as set forth in claim 1 wherein said step of inserting a portion of a wire beneath the selected portion of the patient's skin includes inserting the portion of the wire beneath skin at a depression formed between extensor tendons on a dorsum of the patient's hand, said step of separating connective tissue includes separating connective tissue disposed beneath the skin at the depression formed between bones in the patient's hand, said method further includes moving an implant along the wire to a location beneath skin at the depression formed between bones in the patient's hand.

5. A method as set forth in claim 1 wherein said step of inserting a portion of a wire beneath the selected portion of the patient's skin includes inserting the portion of the wire beneath dimples cellulite in the patient's skin, said step of separating connective tissue includes separating connective tissue disposed beneath the dimples in the patient's skin, said method further includes moving an implant along the wire to a location disposed beneath the dimples in the patient's skin.

6. A method as set forth in claim 1 wherein said step of separating connective tissue includes forming a planar separation beneath the selected portion of the patient's skin, said method further includes the step of moving an implant along the wire into the planar incision and expanding the implant while the implant is in the separation.

7. A method as set forth in claim 6 wherein said step of expanding the implant includes unrolling the implant.

8. A method as set forth in claim 6 wherein said step of expanding the implant includes expanding a plurality of pleats in the implant.

9. A method as set forth in claim 6 wherein said step of expanding the implant includes moving at least one arm connected with the implant relative to the wire.

10. A method as set forth in claim 6 wherein said step of expanding the implant includes pivoting first and second arms about an axis which extends transverse to the wire with the first arm connected to a first portion of the implant and the second arm connected to a second portion of the implant.

11. A method as set forth in claim 1 wherein said step of moving blades in the dissecting device relative to connective tissue includes pivoting a first blade in a first direction about an axis which extends transverse to the wire and pivoting a second blade in a second direction about the axis which extends transverse to the wire.

12. A method as set forth in claim 1 wherein said step of moving blades in the dissecting device includes forming a planar separation beneath the selected portion of the patient's skin.

13. A method as set forth in claim 1 wherein said step of moving blades in the dissecting device includes pushing body tissue aside with the blades to at least partially form a planar separation in the body tissue beneath the selected portion of the patient's skin.

14. A method as set forth in claim 1 wherein said step of moving blades in the dissecting device includes cutting body tissue with the blades to at least partially form a planar separation in the body tissue beneath the selected portion of the patient's skin.

15. A method of altering the appearance of a selected portion of a patient's skin, said method comprising the steps of moving a dissecting device to a location beneath the selected portion of the patient's skin, separating connective tissue which connects the selected portion of the patient's skin, said step of separating connective tissue includes moving blades in the dissecting device relative to the connective tissue to form a planar separation beneath the selected portion of the patient's skin, moving an implant into the planar separation beneath the selected portion of the patient's skin, and expanding the implant while at least a portion of the implant is in the planar separation beneath the selected portion of the patient's skin.

16. A method as set forth in claim 15 wherein said step of expanding the implant includes moving a first portion of the implant in a first direction in the planar separation and moving a second portion of the implant in a second direction in the planar separation.
